(19) United States
(12) Patent Application Publication
(10) Pub. No.: US 2004/0110194 A1
(43) Pub. Date: Jun. 10, 2004

(54) Genes regulated by human cytokines

(75) Inventors: Thierry Sornasse, Mountain View, CA (US); Benjamin Graeme Cocks, Sunnyvale, CA (US); Bharati Sanjawala, Los Altos, CA (US)

Correspondence Address: INCYTE CORPORATION, 3160 PORTER DRIVE, PALO ALTO, CA, 94304, US

(73) Assignee: Incyte Corporation, Palo Alto, CA (US)

(21) Appl. No.: 10/637,855

(22) Filed: Aug. 7, 2003

Related U.S. Application Data
(63) Continuation of application No. 09/435,247, filed on Nov. 5, 1999, now abandoned.

(51) Int. Cl. C12Q 1/68; C07H 21/04; C07K 14/52

(52) U.S. Cl. 435/6; 435/69.1; 435/320.1; 435/325; 536/23.5; 530/351

(57) ABSTRACT

The present invention relates to purified polynucleotides and a composition comprising a plurality of polynucleotide probes that are modulated in response to cytokines and which are associated with human immune response, conditions, disorders, and diseases. The present invention presents methods for using the polynucleotides, and use of the polynucleotide probes as hybridizable elements in a microarray.

This application is a continuation of U.S. application Ser. No. 09/435,247, filed Nov. 5, 1999, which is hereby expressly incorporated by reference.

### FIELD OF THE INVENTION

The present invention relates to a plurality of polynucleotides which may be used in detecting genes modulated in response to human cytokines. In particular, the present invention provides for the use of these polynucleotides in the diagnosis of conditions, disorders, and diseases associated with the immune system and immune response.

### BACKGROUND OF THE INVENTION

Mammalian peripheral blood comprises cells of the erythroid, myeloid, and lymphoid lineages. (See, e.g., Rapaport (1987) Introduction to Hematology, Lipincott, Philadelphia Pa.; Jandi (1987) Blood: Textbook of Immunology, Little, Brown and Co., Boston Mass.; and Paul (1998) Fundamental Immunology (4^{th} ed.), Raven Press, New York N.Y.). Each of these lineages are derived from a pluripotent stem cell which, upon exposure to various molecules and other types of cells, differentiate into effector cells which migrate into the blood and other organs. These cells include red blood cells and platelets (erythroid), macrophages and granulocytes (myeloid), and T and B lymphocytes (lymphoid). The latter two groups of cells mediate immune responses to pathogens such as bacteria, parasites, and viruses.

Functional interaction of the cell types involved in immune responses involves transfer of signals via soluble messenger molecules known as cytokines. Both hematopoietic cells and non-hematopoietic cells produce cytokines which stimulate the activation, differentiation and proliferation of T cells, B cells, macrophages, and granulocytes during an active immune response. Cytokines bind to specific receptors expressed on cellular membranes and transduce a signal through the cell. Depending on the type of cytokine and the cell to which it binds, this signal initiates activation, differentiation, growth, and/or apoptosis (Aggarwal and Gutterman (1991) Human Cytokines: Handbook for Basic and Clinical Research, Blackwell, Oxford, UK).

T cells, which respond to and produce a variety of cytokines, are divided into two major groups, CD4⁺ T helper (Th) cells, and CD8⁺ cytotoxic T lymphocytes (CTL). Immune responses are primarily regulated by CD4⁺ Th cells which fall into two subclasses based on the kinds of cytokines they secrete. Th1 cells secrete primarily IL-2 and IFN-γ, regulate the responses of CTLs, B cells and macrophages, and orchestrate the removal of intracellular pathogens. In contrast, Th2 cells secrete primarily IL4 and IL-10 and promote the development of certain antibody responses such as IgG1, IgA, and IgE, an excess of the latter triggering allergic responses. In addition, Th2 cells remove extracellular pathogens, which include various bacteria and parasites (Morel and Oriss (1998) Crit. Rev. Immunol. 18:275-303).

Further studies have shown that the Th1 cytokine response predominates in organ-specific autoimmune disorders such as insulin-dependent diabetes mellitus (IDDM), multiple sclerosis (MS), rheumatoid arthritis (RA), and Crohn's disease. A Th1 response also predominates in acute allograft rejection, eradication of tumors, and unexplained recurrent abortions. Th2 responses predominate in allergy and other atopic disorders, transplantation tolerance, chronic graft versus host disease (GVHD), and systemic autoimmune disease such as systemic lupus erythmatosus (Romangnani et al. (1997) Int. Arch. Allergy Immunol. 113:153-156).

Genes affected by these molecules may reasonably be expected to be markers of immune cell development, function, and activity. During immune responses, immune cells make a plethora of different cytokines that affect cellular functions. Until now, in vitro studies have focused on the effects of one or two cytokines on gene expression, but have not recreated the complex environment of multiple signals that occur in vivo by studying the effect of multiple cytokines simultaneously. This approach would provide a high throughput method of screening for a cytokine-related disease, assessing the efficacy of treatment for various disorders, conditions, and diseases, and providing information regarding novel genes up- or down-regulated by a complex mixture of cytokines that skew toward a particular immune response.

The present invention provides a method of high-throughput screening using a plurality of probes and purified polynucleotides in a diagnostic context as markers of various immune conditions, diseases, and disorders.

### SUMMARY OF THE INVENTION

The present invention provides a composition comprising a plurality of polynucleotides wherein each polynucleotides comprises at least a fragment of a gene of SEQ ID NOs:1-516 as presented in the Sequence Listing. These polynucleotides are used to assess gene expression which is modulated by cytokines and is associated with an immune response or an immune system disorder. The invention also provides purified polynucleotides wherein each of the polynucleotides comprises at least a fragment of a gene selected from SEQ ID NOs:1-243 or a complement thereof whose expression is modulated by cytokines and is associated with an immune response or an immune system disorder. In one embodiment, each polynucleotide comprises at least a fragment of a gene selected from SEQ ID NOs:1-172 whose transcript level in a sample is altered in response to both pro-inflammatory cytokines such as IL-1β, IL-6, interferon (IFN)-γ, tumor necrosis factor (TNF)-α, IL-18, IL-12, IL-2, and IL-8, and anti-inflammatory cytokines such as IL4, IL-10, IL-13, transforming growth factor (TGF)-β, IL-7, IL-3, IL-5, granulocyte-macrophage colony-stimulating factor (GM-CSF), granulocyte colony stimulating factor (G-CSF), leukemia inhibitory factor (LIF), and leptin. In another embodiment, each polynucleotide comprises at least a fragment of a gene selected from SEQ ID NOs:173-218 whose transcript level in a sample is altered in response to pro-inflammatory cytokines. In another embodiment, each polynucleotide comprises at least a fragment of a gene selected from SEQ ID NOs:219-243 whose transcript level in a sample is altered in response to anti-inflammatory cytokines. In one aspect, the polynucleotides of the composition are immobilized on a substrate.

The invention also provides a high throughput method for detecting a polynucleotide in a sample, the method comprising hybridizing the polynucleotide composition with at least one polynucleotide in the sample, thereby forming a hybridization complex; and detecting the hybridization complex, wherein the presence of the hybridization complex indicates the presence of the polynucleotide in the sample.

The invention also provides a high throughput method of screening a library of molecules or compounds to identify a ligand, the method comprising combining the polynucleotide composition with a library of molecules or compounds under conditions to allow specific binding; and detecting specific binding, thereby identifying a ligand. Libraries of molecules or compounds are selected from DNA molecules, RNA molecules, peptide nucleic acids (PNAs), mimetics, peptides, and proteins. The invention additionally provides a method for purifying a ligand, the method comprising combining a polynucleotide of the invention with a sample under conditions which allow specific binding, recovering the bound polynucleotide, and separating the polynucleotide from the ligand, thereby obtaining purified ligand.

The invention provides an expression vector containing a polynucleotide, a host cell containing the expression vector, and a method for producing a protein comprising culturing the host cell under conditions for the expression of protein and recovering the protein from the host cell culture.

The invention also provides a protein and a method for screening a library of molecules or compounds to identify at least one ligand which specifically binds the protein. The method comprises combining the protein or a portion thereof with the library of molecules or compounds under conditions to allow specific binding and detecting specific binding, thereby identifying a ligand which specifically binds the protein. Libraries of molecules or compounds are selected from DNA molecules, RNA molecules, PNAs, mimetics, peptides, proteins, agonists, antagonists, antibodies or their fragments, immunoglobulins, inhibitors, drug compounds, and pharmaceutical agents. The invention further provides for using a protein to purify a ligand. The method comprises combining the protein or a portion thereof with a sample under conditions to allow specific binding, recovering the bound protein, and separating the protein from the ligand, thereby obtaining purified ligand.

The invention also encompasses a method of screening a patient for an immune response, disorder, condition, or disease comprising obtaining a sample from the patient; contacting the sample with polynucleotides immobilized on a substrate under conditions to allow formation of a hybridization complex; detecting and quantifying hybridization complex to determine hybridization complex level; and comparing hybridization complex level with a standard, wherein a change in hybridization complex level relative to the standard is indicative of the immune disorder, condition, or disease. The immune disorder, condition, or disease includes pro-inflammatory disorders such as viral infections and organ-specific autoimmune disorders, including insulin-dependent diabetes mellitus, multiple sclerosis, rheumatoid arthritis, Crohn's disease and pemphigus vulgaris; and anti-inflammatory disorders such as bacterial and parasitic infections, allergies and other atopic disorders, transplantation tolerance, chronic graft versus host disease, and sytemic autoimmune disease including systemic lupus erythematosus.

### DESCRIPTION OF THE TABLES

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

The Sequence Listing is a compilation of polynucleotides obtained by sequencing clone inserts (isolates) of different cDNAs and identified by hybrid complex formation using the cDNAs as probes on a microarray. Each sequence is identified by a sequence identification number (SEQ ID NO) and by the Incyte clone ID from which it was obtained.

Table 1 lists polynucleotides differentially expressed in peripheral blood mononuclear cells (PBMCs) in response to both pro- and anti-inflammatory cytokines. Columns 1 and 2 show the SEQ ID NO and Incyte clone ID, respectively, for each polynucleotide. Columns 3 and 4 show the differential expression of the gene in PBMCs in response to pro-inflammatory and anti-inflammatory cytokines, respectively.

Table 2 lists polynucleotides differentially expressed in PBMCs in response to pro-inflammatory cytokines. Columns 1 and 2 show the SEQ ID NO and Incyte clone ID, respectively, for each polynucleotide. Columns 3 and 4 show the differential expression of the gene in PBMCs in response to pro-inflammatory and anti-inflammatory cytokines, respectively.

Table 3 lists polynucleotides differentially expressed in PBMCs in response to anti-inflammatory cytokines. Columns 1 and 2 show the SEQ ID NO and Incyte clone ID, respectively, for each polynucleotide. Columns 3 and 4 show the differential expression of the gene in PBMCs in response to pro-inflammatory and anti-inflammatory cytokines, respectively.

Table 4 lists polynucleotides differentially regulated in response to pro-inflammatory cytokines, anti-inflammatory cytokines, or both pro- and anti-inflammatory cytokines. Columns 1 and 2 show the SEQ ID NO and Incyte clone ID, respectively, for each polynucleotide. Columns 3 and 4 show the GenBank hit ID and corresponding GenBank 113 database, respectively, for the top hit identified by BLAST analysis. Column 5 shows the gene description for the polynucleotide. Columns 7 and 8 show the differential expression of the gene in PBMCs in response to pro-inflammatory and anti-inflammatory cytokines, respectively.

### DETAILED DESCRIPTION OF THE INVENTION

Before the nucleic acid sequences and methods are presented, it is to be understood that this invention is not limited to the particular machines, methods, and materials described. Although particular embodiments are described, machines, methods, and materials similar or equivalent to these embodiments may be used to practice the invention. The preferred machines, methods, and materials set forth are not intended to limit the scope of the invention which is limited only by the appended claims.

The singular forms “a”, “an”, and “the” include plural reference unless the context clearly dictates otherwise. All technical and scientific terms have the meanings commonly understood by one of ordinary skill in the art. All publications are incorporated by reference for the purpose of describing and disclosing the cell lines, vectors, and methodologies which are presented and which might be used in connection with the invention. Nothing in the specification is to be construed as an admission that the invention is not entitled to antedate such disclosure by virtue of prior invention.

Definitions

“Amplification” refers to the production of additional copies of a nucleotide sequence and is carried out using polymerase chain reaction (PCR) technologies well known in the art.

“Complementary” describes the relationship between two single-stranded nucleotide sequences that anneal by base-pairing (5′-A-G-T-3′ pairs with its complement 3′-T-C-A-5′).

“Cytokine”, as used herein, refers to a cytokine, chemokine, cytokine-like molecule, or other molecule which elicits an immune response, and includes interleukin (IL)-1β, IL-2, IL-3, IL4, IL-5, IL-6, IL-7, IL-8, IL-10, IL-12, IL-13, IL-18, interferon (IFN)-γ, tumor necrosis factor (TNF)-α, transforming growth factor (TGF)-β, granulocyte-macrophage colony-stimulating factor (GM-CSF), granulocyte colony-stimulatory factor (G-CSF), leukemia-inhibitory factor (LIF), and leptin. “Pro-inflammatory” cytokines include IL-1β, IL-6, IFN-γ, TNF-α, IL-18, IL-12, IL-2, and IL-8. “Anti-inflammatory” cytokines include IL4, IL-10, IL-13, TGF-β, IL-7, IL-3, IL-5, GM-CSF, G-CSF, LIF, and leptin.

“E-value” refers to the statistical probability that a match between two sequences occurred by chance.

“Fragment” refers to an Incyte clone or any part of a polynucleotide which retains a usable, functional characteristic. Useful fragments may be used in hybridization technologies, to identify or purify ligands, or in regulation of replication, transcription or translation.

“Ligand” refers to any molecule, agent, or compound which will bind specifically to a complementary site on a polynucleotide or protein. Such ligands stabilize or modulate the activity of polynucleotides or proteins of the invention and may be composed of at least one of the following: inorganic and organic substances including nucleic acids, proteins, carbohydrates, fats, and lipids.

“Microarray” refers to an ordered arrangement of hybridizable elements on a substrate. The elements are arranged so that there are a “plurality” of elements, preferably more than one element, more preferably at least 100 elements, and even more preferably at least 1,000 elements, and most preferably at least 10,000 on a 1 cm² substrate. The maximum number of elements is unlimited, but is at least 100,000 elements. Furthermore, the hybridization signal from each of the elements is individually distinguishable. In the present and preferred embodiment, the elements comprise polynucleotide probes.

“Oligonucleotide” is substantially equivalent to the terms amplimer, primer, oligomer, element, target, and probe and is preferably single stranded.

“Peptide nucleic acid” (PNA) refers to a DNA mimic in which nucleotide bases are attached to a pseudopeptide backbone to increase stability. PNAs, also designated antigene agents, can prevent gene expression by hybridizing to complementary messenger RNA.

“Polynucleotide” refers to a nucleic acid, oligonucleotide, polynucleotide, or any fragment thereof. It may be DNA or RNA of genomic or synthetic origin, and double-stranded or single-stranded.

“Portion” refers to any part of a protein used for any purpose, but especially for the screening of a library of molecules or compounds to identify those which specifically bind to that portion and for producing antibodies.

“Probe” refers to a probe polynucleotide capable of hybridizing with a target polynucleotide to form a probe/target complex. A “target” refers to a chain of nucleotides to which a probe can hybridize by base pairing. In most instances, the sequences of the probe and target will be complementary (no mismatches) when aligned. In some instances, there may be up to a 10% mismatch.

“Protein” refers to an amino acid sequence, peptide, polypeptide, or protein of either natural or synthetic origin. The protein is not limited to the complete, endogenous amino acid sequence and may be a fragment, epitope, variant, or derivative of a protein.

“Purified” refers to any molecules or compounds that are removed from their natural environment and are isolated or separated, and are at least about 60% free, preferably about 75% free, and most preferably about 90% free, from other components with which they are naturally associated.

“Sample” is used in its broadest sense. A sample may comprise a bodily fluid; an extract from a cell, chromosome, organelle, or membrane isolated from a cell; genomic DNA, RNA, or cDNA in solution or bound to a substrate; a cell; a tissue; a tissue print; and the like.

“Specific binding” refers to a specific interaction between two molecules which is dependent upon a particular structure or molecular side groups. For example, the hydrogen bonding between two single stranded nucleic acids or the binding between a protein/epitope and an agonist, antagonist, or antibody.

“Substrate” refers to any rigid or semi-rigid support to which molecules or compounds are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

The Invention

The present invention provides a composition comprising a plurality of polynucleotide probes, wherein each polynucleotide comprises at least a fragment of a gene whose transcript is modulated by human cytokines. The plurality of probes comprise at least a fragment of the identified and novel polynucleotide sequences, SEQ ID NOs:1-516, as presented in the Sequence Listing. Novel polynucleotides were identified using the composition, wherein each polynucleotide comprises at least a fragment of a gene selected from SEQ ID NOs:1-243 whose transcript is modulated by human cytokines. SEQ ID NOs:1-172 comprise at least a fragment of a gene whose transcript level in a sample is modulated in response to both pro-inflammatory cytokines and anti-inflammatory cytokines as shown in Table 1. SEQ ID NOs:173-218 comprise at least a fragment of a gene whose transcript level in a sample is modulated in response to pro-inflammatory cytokines as shown in Table 2. SEQ ID NOs:219-243 comprise at least a fragment of a gene whose transcript level in a sample is modulated in response to anti-inflammatory cytokines as shown in Table 3.

In a particular embodiment, the probes are arranged on a substrate, preferably a microarray. The microarray can be used for large scale genetic or gene expression analysis of a large number of targets. The microarray can also be used in the diagnosis of diseases and in the monitoring of treatments where altered gene expression is associated with an immune response involving an allergy, a bacterial, viral, or parasitic infection, and the like. Further, the microarray can be employed to investigate an individual's predisposition to an autoimmune disorder including insulin-dependent diabetes mellitus, multiple sclerosis, rheumatoid arthritis, Crohn's disease, systemic lupus erythematosus, and the like.

When the composition of the invention is employed as probes on a microarray, the probes are organized in an ordered fashion so that each element is present at a specified location on the substrate. Because the probes are at specified locations on the substrate, the hybridization patterns and intensities, which together create a unique expression profile, can be interpreted in terms of expression levels of particular genes and can be correlated with a particular metabolic process, condition, disorder, disease, stage of disease, or treatment.

The composition comprising a plurality of probes can also be used to identify or purify a molecule or compound which specifically binds to at least one of the probes. These molecules may be identified from a sample or in high throughput mode from a library of mRNAs, cDNAs, genomic fragments, and the like. Typically, samples or libraries will include targets of diagnostic or therapeutic interest. If nucleic acids in a particular sample enhance the hybridization background, it may be advantageous to remove these nucleic acids. One method for removing additional nucleic acids is by hybridizing the sample with immobilized probes and washing away those nucleic acids that do not form hybridization complexes. At a later point, hybridization complexes can be dissociated, thereby releasing the purified targets.

Method for Selecting Polynucleotides

The polynucleotides which represent genes modulated by cytokines were identified by the following method. Samples were prepared from peripheral blood mononuclear cells (PBMCs) treated with pro-inflammatory or anti-inflammatory cytokines over a defined time course. Gene expression patterns between cytokine-treated and untreated cell samples were compared. The comparisons allowed the identification of genes either upregulated or downregulated in response to each cytokine group and identification of genes either upregulated or downregulated in response to both cytokine groups. SEQ ID NOs:1-516 represent genes modulated by cytokines as identified by differential expression of polynucleotide probes on the substrate. Since polynucleotides are identified solely based on expression levels, it is not essential to know a priori the function of the particular gene. The overall pattern of expression is especially useful in characterizing expression patterns associated with an immune response due to an infection or an autoimmune disorder.

Polynucleotides

The polynucleotides of the invention can be genomic DNA, cDNA, mRNA, or any RNA-like or DNA-like material such as peptide nucleic acids, branched DNAs and the like. Polynucleotide probes can be sense or antisense strand. Where targets are double stranded, probes may be either sense or antisense strands. Where targets are single stranded, probes are complementary single strands.

In one embodiment, polynucleotides are cDNAs. In another embodiment, polynucleotides are plasmids. In the case of plasmids, the sequence of interest is the cDNA insert. The size of the cDNAs may vary and is preferably from 50 to 10,000 nucleotides, more preferably from 50 to 4000 nucleotides, and most preferably about 400 nucleotides in length.

Polynucleotides can be prepared by a variety of synthetic or enzymatic methods well known in the art. Polynucleotides can be synthesized, in whole or in part, using chemical methods well known in the art (Caruthers et al. (1980) Nucleic Acids Symp. Ser. (7):215-233). Alternatively, polynucleotides can be produced enzymatically or recombinantly, by in vitro or in vivo transcription.

Nucleotide analogs can be incorporated into polynucleotide probes by methods well known in the art. The only requirement is that the incorporated nucleotide analogs of the probe must base pair with, target nucleotides. For example, certain guanine nucleotides can be substituted with hypoxanthine which base pairs with cytosine residues. However, these base pairs are less stable than those between guanine and cytosine. Alternatively, adenine nucleotides can be substituted with 2,6-diaminopurine which can form stronger base pairs with thymidine than those between adenine and thymidine.

Additionally, polynucleotides can include nucleotides that have been derivatized chemically or enzymatically. Typical chemical modifications include derivatization with acyl, alkyl, aryl or amino groups.

Polynucleotides probes can be synthesized on a substrate. Synthesis on the surface of a substrate may be accomplished using a chemical coupling procedure and a piezoelectric printing apparatus as described by Baldeschweiler et al. (PCT publication WO95/251116). Alternatively, the probe can be synthesized on a substrate surface using a self-addressable electronic device that controls when reagents are added as described by Heller et al. (U.S. Pat. No. 5,605,662; incorporated herein by reference).

Complementary DNA (cDNA) can be arranged and then immobilized on a substrate. Probes can be immobilized by covalent means such as by chemical bonding procedures or UV. In one such method, a cDNA is bound to a glass surface which has been modified to contain epoxide or aldehyde groups. In another case, a cDNA probe is placed on a polylysine coated surface and then UV cross-linked as described by Shalon et al. (WO95/35505). In yet another method, a DNA is actively transported from a solution to a given position on a substrate by electrical means (Heller et al., supra). Alternatively, probes, clones, plasmids or cells can be arranged on a filter. In the latter case, cells are lysed, proteins and cellular components degraded, and the DNA is coupled to the filter by UV cross-linking.

Furthermore, probes do not have to be directly bound to the substrate, but rather can be bound to the substrate through a linker group. The linker groups are typically about 6 to 50 atoms long to provide exposure of the attached probe. Preferred linker groups include ethylene glycol oligomers, diamines, diacids and the like. Reactive groups on the substrate surface react with a terminal group of the linker to bind the linker to the substrate. The other terminus of the linker is then bound to the probe.

Probes can be attached to a substrate by sequentially dispensing reagents for probe synthesis on the substrate surface or by dispensing preformed DNA fragments to the substrate surface. Typical dispensers include a micropipette delivering solution to the substrate with a robotic system to control the position of the micropipette with respect to the substrate. There can be a multiplicity of dispensers so that reagents can be delivered to the reaction regions efficiently.

Uses of the Polynucleotides

The polynucleotide probes of the present invention may be used for a variety of purposes. For example, the composition of the invention may be used as probes on a microarray. The microarray can be used in high-throughput methods such as for detecting a related polynucleotide in a sample, screening libraries of molecules or compounds to identify a ligand, or diagnosing a particular condition, disease, or disorder associated with an immune response. Alternatively, a polynucleotide complementary to a given sequence of the sequence listing can inhibit or inactivate a therapeutically relevant gene related to the polynucleotide.

Array Analysis

I. Sample Preparation

In order to conduct sample analysis, a sample containing targets is provided. The samples can be any sample containing targets and obtained from any bodily fluid (blood, urine, saliva, phlegm, gastric juices, etc.), cultured cells, biopsies, or other tissue or forensic preparations.

DNA or RNA can be isolated from a sample according to any of a number of methods well known to those of skill in the art. For example, methods of purification of nucleic acids are described in Tijssen (1993) Laboratory Techniques in Biochemistry and Molecular Biology: Hybridization With Nucleic Acid Probes Part I. Theory and Nucleic Acid Preparation, Elsevier Science, New York N.Y.). In one case, total RNA is isolated using TRIZOL reagent (Life Technologies, Gaithersburg Md.), and mRNA is isolated using oligo d(T) column chromatography or glass beads. In one alternative, when targets are derived from an mRNA, targets can be a DNA reverse transcribed from that mRNA, an RNA transcribed from that DNA, a DNA amplified from that DNA, an RNA transcribed from the amplified DNA, and the like. When target is derived from DNA, target can be RNA reverse transcribed from that DNA, or DNA amplified from that DNA. In yet another alternative, targets are prepared by more than one method.

When targets in the sample are amplified it is desirable to maintain their relative abundances, including low abundance transcripts. Total mRNA can be amplified by reverse transcription using a reverse transcriptase and a primer consisting of oligo d(T) and a sequence encoding the phage T7 promoter to provide a single stranded DNA template. The second DNA strand is polymerized using a DNA polymerase and a RNAse which assists in breaking up the DNA/RNA hybrid. After synthesis of the double stranded DNA, T7 RNA polymerase can be added, and RNA transcribed from the second DNA strand template as described by Van Gelder et al. (U.S. Pat. No. 5,545,522; incorporated herein by reference). RNA can be amplified in vitro, in situ or in vivo (See Eberwine, U.S. Pat. No. 5,514,545; incorporated herein by reference).

It is also advantageous to include quantitation controls to assure that amplification and labeling procedures do not change the true abundance of targets in a sample. For this purpose, a sample is spiked with a known amount of a control target, and the composition of probes includes reference probes which specifically hybridize with the control targets. After hybridization and processing, the hybridization signals should reflect accurately the amounts of control target added to the sample.

Prior to hybridization, it may be desirable to fragment the targets. Fragmentation improves hybridization by minimizing secondary structure and cross-hybridization among target nucleic acids in the sample or with noncomplementary probes. Fragmentation can be performed by mechanical or chemical means.

Targets may be labeled with one or more labeling moieties to allow for detection and quantitation of hybridized probe/target complexes. The labeling moieties can include compositions that can be detected by spectroscopic, photochemical, biochemical, bioelectronic, immunochemical, electrical, optical or chemical means. The labeling moieties include radioisotopes, such as ³²P, ³³P or ³⁵S, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like. Exemplary dyes include quinoline dyes, triarylmethane dyes, phthaleins, azo dyes, cyanine dyes, and the like. Preferably, fluorescent markers absorb light above about 300 nm, preferably above 400 nm, and usually emit light at wavelengths at least greater than 10 nm above the wavelength of the light absorbed. Preferred fluorescent markers include fluorescein, phycoerythrin, rhodamine, lissamine, Cy3, and Cy5.

Labeling can be carried out during an amplification reaction, such as by polymerase chain reaction, nick translation, or in vitro transcription reactions. Label can also be incorporated after or without an amplification step, such as by 5′ or 3′-end-labeling reactions. In 5′-end labeling, the 5′ end of the target is dephosphorylated by alkaline phosphatase and then phosphorylated by T4 polynucleotide kinase in the presence of [γ-³²P]ATP. In 3′-end labeling, the label is incorporated by using either terminal transferase or by incubating the target with a labeled oligonucleotide in the presence of T4 RNA ligase.

Alternatively, the labeling moiety can be incorporated after hybridization once a probe/target complex has formed. In one case, biotin is first incorporated during an amplification step as described above. After the hybridization reaction, unbound nucleic acids are rinsed away so that the only biotin remaining bound to the substrate is that attached to targets that are hybridized to probes. Then, an avidin-conjugated fluorophore, such as avidin-phycoerythrin, that binds with high affinity to biotin is added. In another case, the labeling moiety is incorporated by intercalation into preformed target/probe complexes. In this case, an intercalating dye such as a psoralen-linked dye can be employed.

II. Hybridization and Detection

Hybridization allows a denatured polynucleotide probe and a denatured complementary target to form a stable duplex through base pairing. Hybridization methods are well known to those skilled in the art. (See, e.g., Ausubel, et al. (1997) Short Protocols in Molecular Biology, John Wiley & Sons, New York N.Y., Units 2.8-2.11, 3.18-3.19 and 4-6-4.9.) Conditions can be selected for hybridization where completely complementary probe and target can hybridize, i.e., each base pair must interact with its complementary base pair. Alternatively, conditions can be selected where probe and target have mismatches of up to about 10% but are still able to hybridize. Suitable conditions can be selected, for example, by varying the concentrations of salt in the prehybridization, hybridization, and wash solutions or by varying the hybridization and wash temperatures. With some substrates, the temperature can be decreased by adding formamide to the prehybridization and hybridization solutions.

Hybridization can be performed at low stringency with buffers, such as 5×SSC with 1% sodium dodecyl sulfate (SDS) at 60° C., which permits hybridization between probe and target sequences that contain some mismatches to form probe/target complexes. Subsequent washes are performed at higher stringency with buffers such as 0.2×SSC with 0.1% SDS at either 45° C. (medium stringency) or 68° C. (high stringency), to maintain hybridization of only those probe/target complexes that contain completely complementary sequences. Background signals can be reduced by the use of detergents such as SDS, Sarcosyl, or Triton X-100, or a blocking agent, such as salmon sperm DNA.

Hybridization specificity can be evaluated by comparing the hybridization of control probe to target sequences that are added to a sample in a known amount. The control probe may have one or more sequence mismatches compared with the corresponding target. In this manner, it is possible to evaluate whether only complementary targets are hybridizing to the probes or whether mismatched hybrid duplexes are forming.

Hybridization reactions can be performed in absolute or differential hybridization formats. In the absolute hybridization format, targets from one sample are hybridized to microarray elements, and signals detected after hybridization complexes form. Signal strength correlates with target levels in a sample. In the differential hybridization format, differential expression of a set of genes in two biological samples is analyzed. Targets from the two samples are prepared and labeled with different labeling moieties. A mixture of the two labeled targets is hybridized to the microarray elements, and signals are examined under conditions in which the emissions from the two different labels are individually detectable. Probes in the microarray that are hybridized to substantially equal numbers of targets derived from both biological samples give a distinct combined fluorescence (Shalon et al., PCT publication WO95/35505). In a preferred embodiment, the labels are fluorescent labels with distinguishable emission spectra, such as a lissamine conjugated nucleotide analog and a fluorescein conjugated nucleotide analog. In another embodiment Cy3 and Cy5 fluorophores (Amersham Pharmacia Biotech, Piscataway N.J.) are employed.

After hybridization, the microarray is washed to remove nonhybridized polynucleotides, and complex formation between the hybridizable array elements and the targets is examined. Methods for detecting complex formation are well known to those skilled in the art. In a preferred embodiment, the targets are labeled with a fluorescent label, and measurement of levels and patterns of fluorescence indicative of complex formation is accomplished by fluorescence microscopy, preferably confocal fluorescence microscopy. An argon ion laser excites the fluorescent label, emissions are directed to a photomultiplier, and the amount of emitted light is detected and quantitated. The detected signal should be proportional to the amount of probe/target complexes at each position of the microarray. The fluorescence microscope can be associated with a computer-driven scanner device to generate a quantitative two-dimensional image of hybridization intensity. The scanned image is examined to determine the abundance/expression level of hybridized target.

Typically, microarray fluorescence intensities can be normalized to take into account variations in hybridization intensities when more than one microarray is used under similar test conditions. In a preferred embodiment, individual polynucleotide probe/target complex hybridization intensities are normalized using the intensities derived from internal normalization controls contained on each microarray.

III. Screening Assays

Probes may be used to screen a library of molecules or compounds for specific binding affinity. The libraries may be DNA molecules, RNA molecules, PNAs, peptides, proteins such as transcription factors, enhancers, repressors, and other organic or inorganic ligands which regulate activities such as replication, transcription, or translation of polynucleotides in the biological system. The assay involves combining the probe with the library of molecules or compounds under conditions allowing specific binding, and detecting specific binding of a ligand to the probe.

IV. Purification of Ligand

Probes may be used to purify a ligand from a sample. A method for using a probe to purify a ligand would involve combining the probe with a sample under conditions to allow specific binding, detecting specific binding, recovering the bound protein, and using an appropriate agent to separate the polynucleotide from the purified ligand.

Protein Production and Uses

I. Expression of Encoded Proteins

Polynucleotides of the invention may be cloned into a vector and used to express the encoded protein or portions thereof in host cells. The polynucleotides can be engineered by such methods as DNA shuffling (Stemmer and Crameri, U.S. Pat. No. 5,830,721 incorporated by reference herein) and site-directed mutagenesis to create new restriction sites, alter glycosylation patterns, change codon preference to increase expression in a particular host, produce splice variants, extend half-life, and the like. The expression vector may contain transcriptional and translational control elements (promoters, enhancers, specific initiation signals, and 3′ polyadenylation sequence) from various sources which have been selected for their efficiency in a particular host. The vector, polynucleotide, and regulatory elements are combined using in vitro recombinant DNA techniques, synthetic techniques, and/or in vivo genetic recombination techniques well known in the art and described in Sambrook (supra, ch. 4, 8, 16 and 17).

A variety of host systems may be transformed with an expression vector. These include, but are not limited to, bacteria transformed with recombinant bacteriophage, plasmid, or cosmid DNA expression vectors; yeast transformed with yeast expression vectors; insect cell systems transformed with baculovirus expression vectors; plant cell systems transformed with expression vectors containing viral and/or bacterial elements; or animal cell systems (Ausubel supra, Unit 16). For example, an adenovirus transcription/translation complex may be utilized in mammalian cells. After sequences are ligated into the E1 or E3 region of the viral genome, infective virus are used to transform and express the protein in host cells. The Rous sarcoma virus enhancer or SV40 or EBV-based vectors may also be used for high-level protein expression.

Routine cloning, subcloning, and propagation of polynucleotides can be achieved using the multifunctional PBLUESCRIPT vector (Stratagene, La Jolla Calif.) or PSPORT1 plasmid (Life Technologies). Introduction of a nucleic acid sequence into the multiple cloning site of these vectors disrupts the lacZ gene and allows calorimetric screening for transformed bacteria. In addition, these vectors may be useful for in vitro transcription, dideoxy sequencing, single strand rescue with helper phage, and creation of nested deletions in the cloned sequence.

For long term production of recombinant proteins, the vector can be stably transformed into cell lines along with a selectable or visible marker gene on the same or on a separate vector. After transformation, cells are allowed to grow for about 1 to 2 days in enriched media and then are transferred to selective media. Selectable markers, such as antimetabolite, antibiotic, or herbicide resistance genes, confer resistance to the relevant selective agent and allow growth and recovery of cells which successfully express the introduced sequences. Resistant clones identified either by survival on selective media or by the expression of visible markers, such as anthocyanins, green fluorescent protein (GFP), βglucuronidase, luciferase, and the like, may be propagated using culture techniques. Visible markers are also used to quantify the amount of protein expressed by the introduced genes. Verification that the host cell contains the desired polynucleotide is based on DNA-DNA or DNA-RNA hybridizations or PCR amplification techniques.

The host cell may be chosen for its ability to modify a recombinant protein in a desired fashion. Such modifications include acetylation, carboxylation, glycosylation, phosphorylation, lipidation, acylation and the like. Post-translational processing which cleaves a “prepro” form may also be used to specify protein targeting, folding, and/or activity. Different host cells available from the American Type Culture Collection (Manassas Va.) which have specific cellular machinery and characteristic mechanisms for post-translational activities may be chosen to ensure the correct modification and processing of the recombinant protein.

II. Recovery of Proteins from Cell Culture

Heterologous moieties engineered into a vector for ease of purification include glutathione S-transferase (GST), calmodulin binding peptide (CBP), 6-His, FLAG, MYC, and the like. GST, CBP, and 6-His are purified using commercially available affinity matrices such as immobilized glutathione, calmodulin, and metal-chelate resins, respectively. FLAG and MYC are purified using commercially available monoclonal and polyclonal antibodies. A proteolytic cleavage site may be located between the desired protein sequence and the heterologous moiety for ease of separation following purification. Methods for recombinant protein expression and purification are discussed in Ausubel (supra, unit 16) and are commercially available.

III. Screening Assays

A protein or a portion thereof transcribed and translated from a probe may be used to screen libraries of molecules or compounds in any of a variety of screening assays. The protein or portion thereof may be free in solution, affixed to an abiotic or biotic substrate, borne on a cell surface, or located intracellularly. Specific binding between the protein and a ligand may be measured. Depending on the kind of library being screened, the assay may be used to identify DNA, RNA, PNAs, agonists, antagonists, antibodies, immunoglobulins, inhibitors, mimetics, peptides, proteins, drugs, or any other ligand, which specifically binds the protein. One method for high throughput screening using very small assay volumes and very small amounts of test compound is described by Burbaum et al. (U.S. Pat. No. 5,876,946; incorporated herein by reference) which screens large numbers of molecules for enzyme inhibition or receptor binding.

The protein may be used in screening assays of phagemid or B-lymphocyte immunoglobulin libraries to identify antibodies having the desired specificity. Numerous protocols for competitive binding or immunoassays using either polyclonal or monoclonal antibodies with established specificities are well known in the art. Such immunoassays typically involve the measurement of complex formation between the protein and its specific antibody. The method may employ a two-site, monoclonal-based immunoassay utilizing monoclonal antibodies reactive to two non-interfering epitopes or a competitive binding assay (Pound (1998) Immunochemical Protocols, Humana Press, Totowa N.J.).

IV. Purification of a Ligand

The encoded protein or a portion thereof may be used to purify a ligand from a sample. A method for using a protein or a portion thereof to purify a ligand would involve combining the protein or a portion thereof with a sample under conditions to allow specific binding, detecting specific binding between the protein and ligand, recovering the bound protein, and using an appropriate agent to separate the protein from the purified ligand.

V. Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds α-amino and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-α-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivitized amino acids, and washing with dichloromethane and/or N,N-dimethylformamide. The peptide is cleaved between the peptide carboxy terminus and the linker group to yield a peptide acid or amide. (Novabiochem 1997/98 Catalog and Peptide Synthesis Handbook, San Diego Calif., pp. S1-S20). Automated synthesis may also be carried out on machines such as the ABI 431A peptide synthesizer (PE Biosystems, Foster City Calif.). A protein or portion thereof may be substantially purified by preparative high performance liquid chromatography and its composition confirmed by amino acid analysis or by sequencing (Creighton (1984) Proteins, Structures and Molecular Properties, WH Freeman, New York N.Y.).

Preparation of Antibodies

Various hosts including goats, rabbits, rats, mice, humans, and others may be immunized by injection with protein or any portion thereof. Adjuvants such as Freund's, mineral gels, and surface active substances such as lysolecithin, pluronic polyols, polyanions, peptides, oil emulsions, keyhole limpet hemacyanin (KLH), and dinitrophenol may be used to increase immunological response. The oligopeptide, peptide, or portion of protein used to induce antibodies should consist of at least about five amino acids, more preferably ten amino acids, which are identical to a portion of the natural protein. Oligonucleotides may be fused with proteins such as KLH in order to produce antibodies to the chimeric molecule.

Monoclonal antibodies may be prepared using any technique which provides for the production of antibodies by continuous cell lines in culture. These include, but are not limited to, the hybridoma technique, the human B-cell hybridoma technique, and the EBV-hybridoma technique. (Kohler et al. (1975) Nature 256:495497; Kozbor et al. (1985) J. Immunol. Methods 81:31-42; Cote et al. (1983) Proc. Natl. Acad. Sci. 80:2026-2030; and Cole et al. (1984) Mol. Cell Biol. 62:109-120.)

Alternatively, techniques described for the production of single chain antibodies may be adapted, using methods known in the art, to produce epitope specific single chain antibodies. Antibody fragments which contain specific binding sites for epitopes of the mammalian protein may also be generated. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (Huse et al. (1989) Science 246:1275-1281.)

Labeling of Molecules for Assay

A wide variety of labeling moieties and conjugation techniques are known by those skilled in the art and may be used in various nucleic acid, amino acid, and antibody assays. Synthesis of labeled molecules may be achieved using Promega (Madison Wis.) or Amersham Pharmacia Biotech kits for incorporation of a labeled nucleotide such as ³²P-dCTP, Cy3-dCTP or Cy5-dCTP or amino acid such as ³⁵S-methionine. Nucleotides and amino acids may be directly labeled with a variety of substances including fluorescent, chemiluminescent, or chromogenic agents, and the like, by chemical conjugation to amines, thiols and other groups present in the molecules using reagents such as BIODIPY or FITC (Molecular Probes, Eugene Oreg.).

Diagnostics

The polynucleotides, or fragments thereof, may be used to detect and quantify altered gene expression; absence, presence, or excess expression of mRNAs; or to monitor mRNA levels during therapeutic intervention. Conditions, diseases or disorders associated with altered expression include pro-inflammatory disorders such as organ-specific autoimmune disorders including insulin-dependent diabetes mellitus, multiple sclerosis, rheumatoid arthritis, Crohn's disease and pemphigus vulgaris; and anti-inflammatory disorders including allergies and other atopic disorders, transplantation tolerance, chronic graft versus host disease, and sytemic autoimmune diseases such as systemic lupus erythematosus. In addition to disorders, the polynucleotides are useful for monitoring the progression of infectious diseases including, but not limited to, tuberculosis, leprosy, Leishmania, and viral infections such as HIV infection. These polynucleotides can also be utilized as markers of treatment efficacy against the diseases noted above and other immune disorders, conditions, and diseases over a period ranging from several days to months. The diagnostic assay may use hybridization or amplification technology to compare gene expression in a biological sample from a patient to standard samples in order to detect altered gene expression. Qualitative or quantitative methods for this comparison are well known in the art.

For example, the polynucleotide may be labeled by standard methods and added to a biological sample from a patient under conditions for the formation of hybridization complexes. After an incubation period, the sample is washed and the amount of label (or signal) associated with hybridization complexes, is quantified and compared with a standard value. If the amount of label in the patient sample is significantly altered in comparison to the standard value, then the presence of the associated condition, disease or disorder is indicated.

In order to provide a basis for the diagnosis of a condition, disease or disorder associated with gene expression, a normal or standard expression profile is established. This may be accomplished by combining a biological sample taken from normal subjects, either animal or human, with a probe under conditions for hybridization or amplification. Standard hybridization may be quantified by comparing the values obtained using normal subjects with values from an experiment in which a known amount of a substantially purified target sequence is used. Standard values obtained in this manner may be compared with values obtained from samples from patients who are symptomatic for a particular condition, disease, or disorder. Deviation from standard values toward those associated with a particular condition is used to diagnose that condition.

Such assays may also be used to evaluate the efficacy of a particular therapeutic treatment regimen in animal studies and in clinical trial or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to months.

Gene Expression Profiles

A gene expression profile comprises a plurality of probes and a plurality of detectable hybridization complexes, wherein each complex is formed by hybridization of one or more probes to one or more complementary targets in a sample. The polynucleotide composition of the invention is used as probes on a microarray to analyze gene expression profiles. In one embodiment, the microarray is used to monitor the progression of disease. Researchers can assess and catalog the differences in gene expression between healthy and diseased tissues or cells. By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the microarray is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to characterize expression profiles associated with a particular condition, disorder or disease or treatment of the condition, disorder or disease. Novel treatment regimens may be tested in these animal models using microarrays to establish and then follow expression profiles over time. In addition, microarrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

Assays Using Antibodies

Antibodies directed against epitopes on a protein encoded by a polynucleotide of the invention may be used in assays to quantify the amount of protein found in a particular human cell. Such assays include methods utilizing the antibody and a label to detect expression level under normal or disease conditions. The antibodies may be used with or without modification, and labeled by joining them, either covalently or noncovalently, with a labeling moiety.

Protocols for detecting and measuring protein expression using either polyclonal or monoclonal antibodies are well known in the art. Examples include ELISA, RIA, and fluorescent activated cell sorting (FACS). Such immunoassays typically involve the formation of complexes between the protein and its specific antibody and the measurement of such complexes. These and other assays are described in Pound (supra). The method may employ a two-site, monoclonal-based immunoassay utilizing monoclonal antibodies reactive to two non-interfering epitopes, or a competitive binding assay. (See, e.g., Coligan et al. (1997) Current Protocols in Immunology, Wiley-Interscience, New York N.Y.; Pound, supra)

Therapeutics

The polynucleotides of the present invention are useful in antisense technology. Target protein expression is modulated through the specific binding of an antisense probe sequence to a target sequence which either encodes the target protein or directs its expression. The antisense probe can be DNA, RNA, or nucleic acid mimics and analogs. The target sequence can be cellular mRNA and/or genomic DNA and binding of the antisense sequence can affect translation and/or transcription, respectively. (Rossi et al. (1991) Antisense Res. Dev. 1(3):285-288; Lee et al. (1998) Biochemistry 37(3):900-1010; Pardridge et al. (1995) Proc. Nat. Acad. Sci. 92(12):5592-5596; and Nielsen and Haaima (1997) Chem. Soc. Rev. 96:73-78.)

The polynucleotides of the present invention and fragments thereof can be used as antisense sequences to modify the expression of the protein encoded by the polynucleotide. The antisense sequences can be produced ex vivo, for example by using any of the nucleic acid synthesizers or other automated systems known in the art. Antisense sequences can also be produced by in vitro transcription or amplification (Agrawal, supra). In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater et al. (1998) J. Allergy Cli. Immunol. 102(3):469-475; and Scanlon et al. (1995) 9(13): 1288-1296.) Antisense sequences can also be introduced intracellularly through the use of viral vectors, such as retrovirus and adeno-associated virus vectors. (See, e.g., Miller (1990) Blood 76:271; Ausubel, supra; Uckert and Walther (1994) Pharmacol. Ther. 63(3):323-347.) Other gene delivery mechanisms include liposome-derived systems, artificial viral envelopes, and other systems known in the art. (See, e.g., Rossi (1995) Br. Med. Bull. 51(1):217-225; Boado et al. (1998) J. Pharm. Sci. 87(11):1308-1315; and Morris et al. (1997) Nucleic Acids Res. 25(14):2730-2736.)

Molecules which modulate the expression of a polynucleotide of the invention or activity of the encoded protein are useful as therapeutics for conditions and disorders associated with an immune response. Such molecules include agonists which increase the expression or activity of the polynucleotide or encoded protein, respectively; or antagonists which decrease expression or activity of the polynucleotide or encoded protein, respectively. In one aspect, an antibody which specifically binds the protein may be used directly as an antagonist or indirectly as a targeting or delivery mechanism for bringing a pharmaceutical agent to cells or tissues which express the protein.

Additionally, any of the proteins or their ligands, or complementary nucleic acid sequences may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the conditions and disorders associated with an immune response. Using this approach, one may be able to achieve therapeutic efficacy with lower dosages of each agent, thus reducing the potential for adverse side effects. Further, the therapeutic agents may be combined with pharmaceutically-acceptable carriers including excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton Pa.).

It is understood that this invention is not limited to the particular methodology, protocols, and reagents described, as these may vary. It is also understood that the terminology used herein is for the purpose of describing particular embodiments only, and is not intended to limit the scope of the present invention which will be limited only by the appended claims. The examples below are provided to illustrate the subject invention and are not included for the purpose of limiting the invention.

### EXAMPLES

I. Construction of cDNA Libraries

RNA was purchased from CLONTECH Laboratories, Inc. (Palo Alto Calif.) or isolated from various tissues. Some tissues were homogenized and lysed in guanidinium isothiocyanate, while others were homogenized and lysed in phenol or in a suitable mixture of denaturants, such as TRIZOL reagent (Life Technologies). The resulting lysates were centrifuged over CsCl cushions or extracted with chloroform. RNA was precipitated with either isopropanol or ethanol and sodium acetate, or by other routine methods.

Phenol extraction and precipitation of RNA were repeated as necessary to increase RNA purity. In most cases, RNA was treated with DNase. For most libraries, poly(A) RNA was isolated using oligo d(T)-coupled paramagnetic particles (Promega), OLIGOTEX latex particles (QIAGEN, Valencia Calif.), or an OLIGOTEX mRNA purification kit (QIAGEN). Alternatively, poly(A) RNA was isolated directly from tissue lysates using other kits, including the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies) using the recommended procedures or similar methods known in the art. (See Ausubel, supra, Units 5.1 through 6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Pharmacia Biotech) or preparative agarose gel electrophoresis. cDNAs were ligated into compatible restriction enzyme sites of the polylinker of the PBLUESCRIPT plasmid (Stratagene), PSPORT1 plasmid (Life Technologies), or PINCY plasmid (Incyte Pharmaceuticals, Palo Alto Calif.). Recombinant plasmids were transformed into XL1-Blue, XL1-BlueMRF, or SOLR competent E. coli cells (Stratagene) or DH5α, DH10B, or ELECTROMAX DH10B competent E. coli cells (Life Technologies).

In some cases, libraries were superinfected with a 5×excess of the helper phage, M13K07, according to the method of Vieira et al. (1987, Methods Enzymol. 153:3-11) and normalized or subtracted using a methodology adapted from Soares (ura), Swaroop et al. (1991, Nucl. Acids Res. 19:1954), and Bonaldo et al. (1996, Genome Research 6:791-806). The modified Soares normalization procedure was utilized to reduce the repetitive cloning of highly expressed high abundance cDNAs while maintaining the overall sequence complexity of the library. Modification included significantly longer hybridization times which allowed for increased gene discovery rates by biasing the normalized libraries toward those infrequently expressed low-abundance cDNAs which are poorly represented in a standard transcript image (Soares et al. (1994) Proc. Natl. Acad. Sci. 91:9228-9232).

II. Isolation and Sequencing of cDNA Clones

Plasmids were recovered from host cells by in vivo excision using the UNIZAP vector system (Stratagene) or by cell lysis. Plasmids were purified using one of the following: the Magic or WIZARD Minipreps DNA purification system (Promega); the AGTC Miniprep purification kit (Edge BioSystems, Gaithersburg Md.); the QIAWELL 8, QIAWELL 8 Plus, or QIAWELL 8 Ultra plasmid purification systems, or the R.E.A.L. PREP 96 plasmid purification kit (QIAGEN). Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes) and a FLUOROSKAN II fluorescence scanner (Labsystems Oy, Helsinki, Finland).

cDNA sequencing reactions were processed using standard methods or high-throughput instrumentation such as the ABI CATALYST 800 thermal cycler (PE Biosystems) or the DNA ENGINE thermal cycler (MJ Research, Watertown Mass.) in conjunction with the HYDRA microdispenser (Robbins Scientific, Sunnyvale Calif.) or the MICROLAB 2200 system (Hamilton). cDNA sequencing reactions were prepared using reagents provided by Amersham Pharmacia Biotech or supplied in ABI sequencing kits such as the ABI PRISM BIGDYE cycle sequencing kit (PE Biosystems). Electrophoretic separation of cDNA sequencing reactions and detection of labeled polynucleotides were carried out using the MEGABACE 1000 DNA sequencing system (Amersham Pharmacia Biotech); the ABI PRISM 373 or 377 sequencing system (PE Biosystems) in conjunction with standard ABI protocols and base calling software; or other sequence analysis systems known in the art. Reading frames within the cDNA sequences were identified using standard methods (reviewed in Ausubel, supra, Unit 7.7).

III. Extension of cDNA Sequences

Nucleic acid sequences were extended using Incyte cDNA clones and oligonucleotide primers. One primer was synthesized to initiate 5′ extension of the known fragment, and the other, to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed. Preferred libraries are ones that have been size-selected to include larger cDNAs. Also, random primed libraries are preferred because they will contain more sequences with the 5′ and upstream regions of genes. A randomly primed library is particularly useful if an oligo d(T) library does not yield a full-length cDNA.

High fidelity amplification was obtained by PCR using methods well known in the art. PCR was performed in 96-well plates using the DNA ENGINE thermal cycler (PTC-200; MJ Research). The reaction mix contained DNA template, 200 nmol of each primer, reaction buffer containing Mg²⁺, (NH₄)₂SO₄, and β-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B (Incyte Pharmaceuticals): Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ (Stratagene) were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN reagent (Molecular Probes; 0.25% reagent in 1×TE, v/v) and 0.5 μl of undiluted PCR product into each well of an opaque fluorimeter plate (Corning Costar, Acton MA) and allowing the DNA to bind to the reagent. The plate was scanned in a Fluoroskan II (Labsystems Oy) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleic acids were desalted and concentrated, transferred to 384-well plates, digested with CviJI cholera virus endonuclease (Molecular Biology Research, Madison Wis.), and sonicated or sheared prior to religation into pUC18 vector (Amersham Pharmacia Biotech). For shotgun sequencing, the digested nucleic acids were separated on low concentration (0.6 to 0.8%) agarose gels, fragments were excised, and agar digested with AGARACE enzyme (Promega). Extended clones were religated using T4 DNA ligase (New England Biolabs, Beverly Mass.) into pUC18 vector (Amersham Pharmacia Biotech), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2×carbenicillin liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified using PICOGREEN reagent (Molecular Probes) as described above. Samples with low DNA recoveries were reamplified using the same conditions described above. Samples were diluted with 20% dimethylsulfoxide (DMSO; 1:2, v/v), and sequenced using DYENAMIC energy transfer sequencing primers and the DYENAMIC DIRECT cycle sequencing kit (Amersham Pharmacia Biotech) or the ABI PRISM BIGDYE terminator cycle sequencing kit (PE Biosystems).

IV. Assembly and Analysis of Sequences

Component nucleotide sequences from chromatograms were subjected to PHRED analysis (Phil's Revised Editing Program; Phil Green, University of Washington, Seattle Wash.) and assigned a quality score. The sequences having at least a required quality score were subject to various pre-processing algorithms to eliminate low quality 3′ ends, vector and linker sequences, polyA tails, Alu repeats, mitochondrial and ribosomal sequences, bacterial contamination sequences, and sequences smaller than 50 base pairs.

Sequences were screened using the BLOCK 2 program (Incyte Pharmaceuticals), a motif analysis program based on sequence information contained in the SWISS-PROT and PROSITE databases (Bairoch et al. (1997) Nucleic Acids Res. 25:217-221; Attwood et al. (1997) J. Chem. Inf. Comput. Sci. 37:417-424).

Processed sequences were subjected to assembly procedures in which the sequences were assigned to bins, one sequence per bin. Sequences in each bin were assembled to produce consensus sequences, templates. Subsequent new sequences were added to existing bins using the Basic Local Alignment Search Tool (BLAST; Altschul (1993) J. Mol. Evol. 36:290-300; Altschul et al. (1990) J. Mol. Biol. 215:403410; Karlin et al. (1988) Proc. Natl. Acad. Sci. 85:841-845), BLASTn (v.1.4, WashU), and CROSSMATCH software (Phil Green, supra). Candidate pairs were identified as all BLAST hits having a quality score greater than or equal to 150. Alignments of at least 82% local identity were accepted into the bin. The component sequences from each bin were assembled using PHRAP (Phil's Revised Alignment Program; Phil Green, supra). Bins with several overlapping component sequences were assembled using DEEP PHRAP (Phil Green, supra).

Bins were compared against each other, and those having local similarity of at least 82% were combined and reassembled. Reassembled bins having templates of insufficient overlap (less than 95% local identity) were re-split. Assembled templates were also subjected to analysis by STITCHER/EXON MAPPER algorithms which analyzed the probabilities of the presence of splice variants, alternatively spliced exons, splice junctions, differential expression of alternative spliced genes across tissue types, disease states, and the like. These resulting bins were subjected to several rounds of the above assembly procedures to generate the template sequences found in the LIFESEQ GOLD database (Incyte Pharmaceuticals).

The assembled templates were annotated using the following procedure. Template sequences were analyzed using BLASTn (v2.0, NCBI) versus GBpri (GenBank version 109). “Hits” were defined as an exact match having from 95% local identity over 200 base pairs through 100% local identity over 100 base pairs, or a homolog match having an E-value of ≦1×10⁻⁸. The hits were subjected to frameshift FASTx versus GENPEPT (GenBank version 109). In this analysis, a homolog match was defined as having an E-value of ≦1×10⁻⁸. The assembly method used above was described in “Database and System for Storing, Comparing and Displaying Related Biomolecular Sequence Information,” U.S. Ser. No. 09/276,534, filed Mar. 25, 1999, incorporated by reference herein, and the LIFESEQ GOLD user manual (Incyte Pharmaceuticals).

Following assembly, template sequences were subjected to motif, BLAST, Hidden Markov Model (HMM; Pearson and Lipman (1988) Proc. Natl. Acad. Sci. 85:2444-2448; Smith and Waterman (1981) J. Mol. Biol. 147:195-197), and functional analyses, and categorized in protein hierarchies using methods described in “Database System Employing Protein Function Hierarchies for Viewing Biomolecular Sequence Data,” U.S. Ser. No. 08/812,290, filed Mar. 6, 1997; “Relational Database for Storing Biomolecule Information,” U.S. Ser. No. 08/947,845, filed Oct. 9, 1997; “Project-Based Full-Length Biomolecular Sequence Database,” U.S. Pat. No. 5,953,727; and “Relational Database and System for Storing Information Relating to Biomolecular Sequences,” U.S. Ser. No. 09/034,807, filed Mar. 4, 1998, all of which are incorporated by reference herein. Template sequences may be further queried against public databases such as the GenBank rodent, mammalian, vertebrate, eukaryote, prokaryote, and human EST databases.

V. Preparation of Microarrays

The polynucleotides present on the human GENEALBUM GEM series 1-6 microarrays (Incyte Pharmaceuticals) represent template sequences derived from the LIFESEQ GOLD assembled human sequence database (Incyte Pharmaceuticals). In cases where more than one clone was available for a particular template, the 5′-most clone in the template was used on the microarray. Polynucleotides were amplified from bacterial cells using primers complementary to vector sequences flanking the cDNA insert. Thirty cycles of PCR increased the initial quantity of polynucleotide from 1-2 ng to a final quantity greater than 5 μg. Amplified polynucleotides were then purified using SEPHACRYL-400 columns (Amersham Pharmacia Biotech).

Purified polynucleotides were immobilized on polymer-coated glass slides. Glass microscope slides (Corning, Corning N.Y.) were cleaned by ultrasound in 0.1% SDS and acetone, with extensive distilled water washes between and after treatments. Glass slides were etched in 4% hydrofluoric acid (VWR Scientific Products Corporation, West Chester Pa.), washed extensively in distilled water, and coated with 0.05% aminopropyl silane (Sigma, St. Louis Mo.) in 95% ethanol. Coated slides were cured in a 110° C oven. polynucleotides were applied to the coated glass substrate using a procedure described in U.S. Pat. No. 5,807,522, incorporated herein by reference. One microliter of the polynucleotide at an average concentration of 100 ng/ul was loaded into the open capillary printing element by a high-speed robotic apparatus which then deposited about 5 nl of polynucleotide per slide.

Microarrays were V-crosslinked using a STRATALINKER UV-crosslinker (Stratagene), and then washed at room temperature once in 0.2% SDS and three times in distilled water. Non-specific binding sites were blocked by incubation of microarrays in 0.2% casein in phosphate buffered saline (Tropix, Bedford Mass.) for 30 minutes at 60° C. followed by washes in 0.2% SDS and distilled water as before.

VI. Preparation of Target Polynucleotides

Cytokine Treatment of PBMCs

Peripheral blood mononuclear cells (PBMCs) were isolated from freshly obtained peripheral blood of two healthy donors by centrifugation of the lymphocyte enriched blood fraction over a HYPAQUE ficoll gradient (Sigma). The isolated PBMCs were grown in Yssel's media (Yssel (1984) J. Immunol. Methods 72:219-225) supplemented with 1% pooled type AB human serum. About 2×10⁷ .PBMCs from each donor were treated with Group A (pro-inflammatory) cytokines for two hours at 37° C., at the following concentrations: IL-1β, at 10 ng/ml (R&D Systems, Minneapolis Minn.); IL-2 at 10 ng/mil (R&D Systems); IL-6 at 10 ng/ml (R&D Systems); IL-8 at 10 ng/ml (R&D Systems); IL-12 at 1 ng/ml (R&D Systems); IL-18 at 10 ng/ml (Peprotech, Inc., Rockyhill N.J.); TNFα at 10 ng/ml (R&D Systems); and IFNγ at 50 ng/ml (R&D Systems). Similarly, 2×10⁷ PBMCs from each donor were treated with Group B (anti-inflammatory) cytokines for two hours at 37° C., using the following concentrations: IL-3 at 10 ng/ml (R&D Systems); IL-4 at 10 ng/ml (R&D Systems); IL-5 at 10 ng/ml (R&D Systems); IL-7 at 10 ng/ml (R&D Systems); IL-10 at 50 ng/ml (R&D Systems); LIF at 20 ng/ml (R&D Systems); GM-CSF at 10 ng/ml (R&D Systems); G-CSF at 100 ng/ml (R&D Systems); TGFβ at 10 ng/ml (R&D Systems); and leptin at 100 nM (Peprotech). Approximately 1×10⁸ PBMCs from each donor were untreated controls.

Isolation and Labeling of Target Polynucleotides

Cells were harvested and lysed in TRIZOL reagent (5×10⁶ cells/1 ml; Life Technologies). Cell lysates were vortexed, incubated at room temperature for 2-3 minutes, and extracted with 0.5 ml chloroform. The extract was mixed, incubated at room temperature for 5 minutes, and centrifuged at 16,000 g for 15 minutes at 4° C. The aqueous layer was collected and an equal volume of isopropanol was added. Samples were mixed, incubated at room temperature for 10 minutes, and centrifuged at 16,000 g for 20 minutes at 4° C. The supernatant was removed and the RNA pellet was washed with 70% ethanol, centrifuged at 16,000 g at 4° C., and resuspended in RNase-free water. The concentration of RNA was determined by measuring the optical density at 260 nm.

Poly(A) RNA was prepared using an OLIGOTEX mRNA kit (QIAGEN) with the following modifications: OLIGOTEX beads were washed in tubes instead of on spin columns, resuspended in elution buffer, and then loaded onto spin columns to recover mRNA. To obtain maximum yield, the mRNA was eluted twice. Each poly(A) RNA sample was reverse transcribed using MMLV reverse-transcriptase, 0.05 pg/μl oligo-dT primer (21 mer), 1×first strand buffer, 0.03 units/ul RNase inhibitor, 500 uM dATP, 500 uM dGTP, 500 uM dTTP, 40 uM dCTP, and 40 uM either dCTP-Cy3 or dCTP-Cy5 (Amersham Pharmacia Biotech). The reverse transcription reaction was performed in a 25 ml volume containing 200 ng poly(A) RNA using the GEMBRIGHT kit (Incyte Pharmaceuticals). Specific control poly(A) RNAs (YCFR06, YCFR45, YCFR67, YCFR85, YCFR43, YCFR22, YCFR23, YCFR25, YCFR44, YCFR26) were synthesized by in vitro transcription from non-coding yeast genomic DNA (W. Lei, unpublished). As quantitative controls, control mRNAs (YCFR06, YCFR45, YCFR67, and YCFR85) at 0.002 ng, 0.02 ng, 0.2 ng, and 2 ng were diluted into reverse transcription reaction at ratios of 1:100,000, 1:10,000, 1:1000, 1:100 (w/w) to sample mRNA, respectively. To sample differential expression patterns, control mRNAs (YCFR43, YCFR22, YCFR23, YCFR25, YCFR44, YCFR26) were diluted into reverse transcription reaction at ratios of 1:3, 3:1, 1:10, 10:1, 1:25, 25:1 (w/w) to sample mRNA. Reactions were incubated at 37° C. for 2 hr, treated with 2.5 ml of 0.5M sodium hydroxide, and incubated for 20 minutes at 85° C. to the stop the reaction and degrade the RNA.

Targets were purified using two successive CHROMA SPIN 30 gel filtration spin columns (CLONTECH). Cy3- and Cy5-labeled reaction samples were combined as described below and ethanol precipitated using 1 ml of glycogen (1 mg/ml), 60 ml sodium acetate, and 300 ml of 100% ethanol. The target was then dried to completion using a SpeedVAC system (Savant Instruments Inc., Holbrook N.Y.) and resuspended in 14 μL 5×SSC/0.2% SDS.

VII. Hybridization and Detection

Hybridization reactions contained 9 μl of target mixture consisting of 0.2 μg each of Cy3 and Cy5 labeled cDNA synthesis products in 5×SSC, 0.2% SDS hybridization buffer. The targets were assigned the following designations: a) a control experiment where the Cy3 and Cy5 targets were cDNA from untreated PBMCs; b) Cy3 was cDNA from untreated PBMCs and Cy5 was cDNA from Group A treated PBMCs; and c) Cy3 was cDNA from untreated PBMCs and Cy5 was cDNA from Group B treated PBMCs. The target mixture was heated to 65° C. for 5 minutes and was aliquoted onto the microarray surface and covered with an 1.8 cm² coverslip. The microarrays were transferred to a waterproof chamber having a cavity just slightly larger than a microscope slide. The chamber was kept at 100% humidity internally by the addition of 140 μl of 5×SSC in a corner of the chamber. The chamber containing the microarrays was incubated for about 6.5 hours at 60° C. The microarrays were washed for 10 min at 45° C. in low stringency wash buffer (1×SSC, 0.1% SDS), three times for 10 minutes each at 45° C. in high stringency wash buffer (0.1×SSC), and dried.

Reporter-labeled hybridization complexes were detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light was focused on the microarray using a 20×microscope objective (Nikon, Inc., Melville N.Y.). The slide containing the microarray was placed on a computer-controlled X-Y stage on the microscope and raster-scanned past the objective. The 1.8 cm×1.8 cm microarray used in the present example was scanned with a resolution of 20 micrometers.

In two separate scans, the mixed gas multiline laser excited the two fluorophores sequentially. Emitted light was split, based on wavelength, into two photomultiplier tube detectors (PMT R1477; Hamamatsu Photonics Systems, Bridgewater N.J.) corresponding to the two fluorophores. Appropriate filters positioned between the microarray and the photomultiplier tubes were used to filter the signals. The emission maxima of the fluorophores used were 565 nm for Cy3 and 650 nm for Cy5. Each microarray was typically scanned twice, one scan per fluorophore using the appropriate filters at the laser source, although the apparatus was capable of recording the spectra from both fluorophores simultaneously.

The sensitivity of the scans was calibrated using the signal intensity generated by a cDNA control species. Samples of the calibrating cDNA were separately labeled with the two fluorophores and identical amounts of each were added to the hybridization mixture. A specific location on the microarray contained a complementary DNA sequence, allowing the intensity of the signal at that location to be correlated with a weight ratio of hybridizing species of 1:100,000.

The output of the photomultiplier tube was digitized using a 12-bit RTI-835H analog-to-digital (A/D) conversion board (Analog Devices, Inc., Norwood, Mass.) installed in an IBM-compatible PC computer. The digitized data were displayed as an image where the signal intensity was mapped using a linear 20-color transformation to a pseudocolor scale ranging from blue (low signal) to red (high signal). The data was also analyzed quantitatively. Where two different fluorophores were excited and measured simultaneously, the data were first corrected for optical crosstalk (due to overlapping emission spectra) between the fluorophores using each fluorophore's emission spectrum.

A grid was superimposed over the fluorescence signal image such that the signal from each spot was centered in each element of the grid. The fluorescence signal within each element was then integrated to obtain a numerical value corresponding to the average intensity of the signal. The software used for signal analysis was the GEMTOOLS gene expression analysis program (Incyte Pharmaceuticals).

VIII. Data Analysis and Results

Genes which exhibited a ≧2-fold change in expression in cytokine-treated vs untreated controls and displayed a signal intensity over 300 were identified using the GEMTOOLS program (Incyte Pharmaceuticals). The polynucleotides comprising SEQ ID NOs:1-516 as presented in the Sequence Listing showed at least a 2-fold change in expression in response to pro-inflammatory cytokines, anti-inflammatory cytokines, or both pro-and anti-inflammatory cytokines. Comparisons of expression between two different cytokine pools allowed the identification of genes useful in diagnosing a condition associated with pro-inflammatory response such as organ-specific autoimmune disorders including insulin-dependent diabetes mellitus, multiple sclerosis, rheumatoid arthritis, Crohn's disease and pemphigus vulgaris; anti-inflammatory response such as bacterial and parasitic infections, allergies and other atopic disorders, transplantation tolerance, chronic graft versus host disease, and sytemic autoimmune disease including systemic lupus erythematosus; or an immune response encompassing characteristics of both pro- and anti-inflammatory response.

Tables 1-4 represent various combinations of the polynucleotides of SEQ ID Nos:1-516 that were up or down regulated at least 2-fold in PBMCs in response to human cytokines. Since the polynucleotides were identified solely based on differential expression in cytokine-treated versus untreated tissue, it is not essential to know a priori the name, structure, or function of a particular gene or protein. The usefulness of the human sequences exists in their immediate value as diagnostics for immune response and immune disorders.

In tables 1-3, columns 1 and 2 list the SEQ ID NO and Incyte clone number, respectively, for the polynucleotides of the invention. Columns 3 and 4 indicate the differential expression of the gene measured at the end of the experiment for pro- and anti-inflammatory cytokine treatment, respectively. Differential expression values are reported as logₙ [control (untreated)÷cytokine-treated]. A value of −1 indicates a 2-fold increase in expression in response to cytokine treatment.

Table 1 lists novel polynucleotides differentially regulated at least 2-fold in response to both pro- and anti-inflammatory cytokines. These genes are associated with the general response of PBMCs to signals from the immune system and the infective process.

Table 2 lists novel polynucleotides differentially regulated at least 2-fold in response to pro-inflammatory cytokines. These genes reflect the response of PBMCs to the mileau of cytokines released during inflammation and represent potentially useful markers for viral infections and autoimmune disorders.

Table 3 lists novel polynucleotides differentially regulated at least 2-fold in response to anti-inflammatory cytokines. These genes reflect the response of PBMCs to the mileau of cytokines released in opposition of an inflammatory response and represent potentially useful markers for bacterial and parasitic infections and allergic response.

Table 4 lists known polynucleotides differentially regulated at least 2-fold in response to pro-inflammatory cytokines, anti-inflammatory cytokines, or both pro- and anti-inflammatory cytokines. Some genes identified in table 4, such as the p53 binding protein 53BP2, IFN-γ accessory factor AF-1, and IL-2 receptor, were previously known to be modulated by cytokines. Other genes identified in table 4, such as thrombomodulin, the mucin-like hormone receptor EMR1, and the LIM protein ESP 1/CRP2, were not previously known to be modulated by cytokines. Columns 1 and 2 list the SEQ ID NO and Incyte clone ID, respectively, for the polynucleotides of the invention. Column 3 provides a description of the gene. Sequences not identified by BLAST are indicated as “Incyte EST”. Columns 4 and 5 show the GenBank ID and corresponding GenBank 113 database, respectively, of the closest homolog identified by BLAST. Columns 6 and 7 indicate the differential expression of the gene measured at the end of the experiment for pro- and anti-inflammatory cytokine treatment, respectively. Differential expression values are reported as logn [untreated÷cytokine-treated].

The polynucleotides of the Sequence Listing have been prepared by current, state-of-the-art, automated methods and, as such, may contain occasional sequencing errors or unidentified nucleotides. Such unidentified nucleotides are designated by an ‘n’. These infrequent unidentified bases do not represent a hindrance to practicing the invention for those skilled in the art. Several methods employing standard recombinant techniques may be used to correct errors and complete the missing sequence information. (See, e.g., those described in Ausubel et al. (1997) Short Protocols in Molecular Biology, John Wiley & Sons, New York N.Y.; and Sambrook et al. (1989) Molecular Cloning. A Laboratory Manual, Cold Spring Harbor Press, Plainview N.Y.)

IX. Complementary Nucleic Acid Molecules

Molecules complementary to the polynucleotide or a fragment thereof are used to detect, decrease, or inhibit gene expression. Although use of oligonucleotides comprising from about 15 to about 30 base pairs is described, the same procedure is used with larger or smaller fragments or their derivatives (PNAs). Oligonucleotides are designed using vector NTI software (Informax, N. Bethesda Md.) and SEQ ID NOs:1-516. To inhibit transcription by preventing promoter binding, a complementary oligonucleotide is designed to bind to the most unique 5′ sequence, most preferably about 10 nucleotides before the initiation codon of the open reading frame. To inhibit translation, a complementary oligonucleotide is designed to prevent ribosomal binding to the mRNA encoding the protein.

X. Probe Preparation, Target Labeling, and Hybridization Analyses

Probe nucleic acid molecules are isolated and applied to a substrate for standard hybridization protocols by one of the following methods. A mixture of probes is fractionated by electrophoresis through an 0.7% agarose gel in 1×TAE [Tris-acetate-EDTA] running buffer and transferred to a nylon membrane by capillary transfer using 20×SSC. Alternatively, the probes are individually ligated to a vector and inserted into bacterial host cells to form a library. Probes are then arranged on a substrate by one of the following methods. In the first method, bacterial cells containing individual clones are robotically picked and arranged on a nylon membrane. The membrane is placed on bacterial growth medium, LB agar containing carbenicillin, and incubated at 37° C. for 16 hours. Bacterial colonies are denatured, neutralized, and digested with proteinase K. Nylon membranes are exposed to UV irradiation in a STRATALINKER UV-crosslinker (Stratagene) to cross-link probe to the membrane.

In the second method, probes are amplified from bacterial vectors by thirty cycles of PCR using primers complementary to vector sequences flanking the insert. Amplified probes are purified using SEPHACRYL400 beads (Amersham Pharmacia Biotech). Purified probes are robotically arrayed onto a glass microscope slide (Corning Science Products, Corning N.Y.). The slide was previously coated with 0.05% aminopropyl silane (Sigma-Aldrich, St. Louis Mo.) and cured at 110° C. The arrayed glass slide (microarray) was exposed to UV irradiation in a STRATALINKER UV-crosslinker (Stratagene).

cDNA targets are made from mRNA templates. Five micrograms of mRNA is mixed with 1 μg random primer (Life Technologies), incubated at 70° C. for 10 minutes, and lyophilized. The lyophilized sample is resuspended in 50 μl of 1×first strand buffer (cDNA Synthesis systems; Life Technologies) containing a dNTP mix, [α-³²P]dCTP, dithiothreitol, and MMLV reverse transcriptase (Stratagene), and incubated at 42° C. for 1-2 hours. After incubation, the target is diluted with 42 μl dH₂O, heated to 95° C. for 3 minutes, and cooled on ice. mRNA in the target is removed by alkaline degradation. The target is neutralized, and degraded mRNA and unincorporated nucleotides are removed using a PROBEQUANT G-50 microcolumn (Amersham Pharmacia Biotech). Targets can be labeled with fluorescent markers, Cy3-dCTP or Cy5-dCTP (Amersham Pharmacia Biotech), in place of the radionucleotide, [³²P]dCTP.

Hybridization is carried out at 65° C. in a hybridization buffer containing 0.5 M sodium phosphate (pH 7.2), 7% SDS, and 1 mM EDTA. After the substrate is incubated in hybridization buffer at 65° C. for at least 2 hours, the buffer is replaced with 10 ml of fresh buffer containing the targets. After incubation at 65° C. for 18 hours, the hybridization buffer is removed, and the substrate is washed sequentially under increasingly stringent conditions, up to 40 mM sodium phosphate, 1% SDS, 1 mM EDTA at 65° C. To detect signal produced by a radiolabeled target hybridized on a membrane, the substrate is exposed to a PHOSPHORIMAGER cassette (Amersham Pharmacia Biotech), and the image is analyzed using IMAGEQUANT data analysis software (Amersham Pharmacia Biotech). To detect signals produced by a fluorescent target hybridized on a microarray, the substrate is examined by confocal laser microscopy, and images are collected and analyzed using GEMTOOLS gene expression analysis software (Incyte Pharmaceuticals).

XI. Expression of the Encoded Protein

Expression and purification of a protein encoded by a polynucleotide of the invention is achieved using bacterial or virus-based expression systems. For expression in bacteria, cDNA is subcloned into a vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into bacterial hosts, such as BL21(DE3). Antibiotic resistant bacteria express the protein upon induction with isopropyl beta-D-thiogalactopyranoside (IPTG). Expression in eukaryotic cells is achieved by infecting Spodoptera frugiperda (Sf9) insect cells with recombinant baculovirus, Autographica californica nuclear polyhedrosis virus. The polyhedrin gene of baculovirus is replaced with the polynucleotide by either homologous recombination or bacterial-mediated transposition involving transfer plasmid intermediates. Viral infectivity is maintained and the strong polyhedrin promoter drives high levels of polynucleotide transcription.

Protein is synthesized as a fusion protein with glutathione S-transferase (GST) permitting rapid, single-step, affinity-based purification of recombinant fusion protein from crude cell lysates. GST enables the purification of fusion proteins on immobilized glutathione under conditions that maintain protein activity and antigenicity (Amersham Pharmacia Biotech). Following purification, the GST moiety is proteolytically cleaved from the protein at specifically engineered sites.

XII. Production of Specific Antibodies

Protein encoded by a polynucleotide of the invention is purified using polyacrylamide gel electrophoresis and used to immunize mice or rabbits. Alternatively, the amino acid sequence of the protein is analyzed using LASERGENE software (DNASTAR) to determine regions of high immunogenicity. An immunogenic epitope near the C-terminus or in a hydrophilic region is selected, synthesized, and used to raise antibodies. Typically, epitopes of about 15 residues in length are produced using an ABI 431A peptide synthesizer (PE Biosystems) using Fmoc-chemistry and coupled to KLH (Sigma-Aldrich) by reaction with N-maleimidobenzoyl-N-hydroxysuccinimide ester to increase immunogenicity.

Rabbits are immunized with the epitope-KLH complex in complete Freund's adjuvant. Immunizations are repeated at intervals in incomplete Freund's adjuvant. After a minimum of seven weeks for mouse or twelve weeks for rabbit, antisera are drawn and tested for antipeptide activity. Testing involves binding the peptide to plastic, blocking with 1% bovine serum albumin, reacting with rabbit antisera, washing, and reacting with radio-iodinated goat anti-rabbit IgG. Antibody titer is then determined.

XIII. Purification of Naturally Occurring Protein Using Specific Antibodies

Naturally occurring or recombinant protein is substantially purified by immunoaffinity chromatography using antibodies specific for the protein. An immunoaffinity column is constructed by covalently coupling the antibody to CNBr-activated SEPHAROSE resin (Amersham Pharmacia Biotech). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIV. Screening Molecules for Specific Binding with the Probe or Protein

The polynucleotide or fragments thereof are labeled with ³²P-dCTP, Cy3-dCTP, Cy5-dCTP (Amersham Pharmacia Biotech), or the protein or portions thereof are labeled with BIODIPY or FITC (Molecular Probes). Libraries of candidate molecules previously arranged on a substrate are incubated in the presence of labeled probe or protein. After incubation under conditions for either a nucleic acid or amino acid sequence, the substrate is washed, and any position on the substrate retaining label, which indicates specific binding or complex formation, is assayed, and the binding molecule is identified. Data obtained using different concentrations of the probe or protein are used to calculate affinity between the labeled probe or protein and the bound molecule.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention which are obvious to those skilled in the field of molecular biology or related fields are intended to be within the scope of the following claims.

What is claimed is:

1. A composition comprising a plurality of polynucleotides whose expression is modulated by cytokines, wherein the polynucleotides comprise SEQ ID NOs:1-516 or a complement thereof.

2. A substantially purified polynucleotide whose expression is modulated by cytokines comprising at least a fragment of a gene selected from SEQ ID NOs:1-243 or a complement thereof.

3. The polynucleotide of claim 2 whose expression is modulated by pro-inflammatory and anti-inflammatory cytokines, wherein the polynucleotide is selected from SEQ ID NOs:1-172 or a complement thereof.

4. The polynucleotide of claim 2 whose expression is modulated by pro-inflammatory cytokines, wherein the polynucleotide is selected from SEQ ID NOs:173-218 or a complement thereof.

5. The polynucleotide of claim 2 whose expression is modulated by anti-inflammatory cytokines, wherein the polynucleotide is selected from SEQ ID NOs:219-243 or a complement thereof.

6. The composition of claim 1, wherein the polynucleotides are immobilized on a substrate.

7. A high throughput method for detecting a polynucleotide in a sample, the method comprising:
(a) hybridizing the composition of claim 1 with the sample, thereby forming hybridization complex; and
(b) detecting the hybridization complex, wherein the presence of the hybridization complex indicates the presence of the polynucleotide in the sample.

8. A high throughput method of screening a library of molecules or compounds to identify a ligand, the method comprising:
(a) combining the composition of claim 1 with a library of molecules or compounds under conditions to allow specific binding; and
(b) detecting specific binding, thereby identifying a ligand.

9. The method of claim 8 wherein the library is selected from DNA molecules, RNA molecules, peptide nucleic acids, mimetics, peptides, and proteins.

10. A method of purifying ligands, the method comprising:
a) combining the polynucleotide of claim 2 with a sample under conditions which allow specific binding;
b) recovering the bound polynucleotide, and
c) separating the polynucleotide from the ligand, thereby obtaining purified ligand.

11. An expression vector containing the polynucleotide of claim 2.

12. A host cell containing the expression vector of claim 11.

13. A method for producing a protein, the method comprising the steps of:
(a) culturing the host cell of claim 11 under conditions for the expression of protein; and
(b) recovering the protein from the host cell culture.

14. A protein or a portion thereof produced by the method of claim 13.

15. A method for screening a library of molecules or compounds to identify at least one ligand which specifically binds a protein, the method comprising:
(a) combining the protein or a portion thereof of claim 14 with the library of molecules or compounds under conditions to allow specific binding; and
(b) detecting specific binding, thereby identifying a ligand which specifically binds the protein.

16. The method of claim 15 wherein the library is selected from DNA molecules, RNA molecules, PNAs, mimetics, peptides, proteins, agonists, antagonists, antibodies or their fragments, immunoglobulins, inhibitors, drug compounds, and pharmaceutical agents.

17. A method of purifying a ligand, the method comprising:
a) combining the protein or a portion thereof of claim 14 with a sample under conditions to allow specific binding;
b) recovering the bound protein; and
c) separating the protein from the ligand, thereby obtaining purified ligand.

18. A method of screening a sample from a patient for an immune response, disorder, condition, or disease, the method comprising:
a) contacting the sample with the composition of claim 1 immobilized on a substrate under conditions to allow formation of a hybridization complex;
b) detecting and quantifying complex formation; and
c) comparing complex formation with a standard, wherein a change complex formation indicates the presence of the immune disorder, condition, or disease.

19. The method of claim 18, wherein the immune disorder, condition, or disease is a pro-inflammatory disorder selected from viral infections, rheumatoid arthritis, insulin-dependent diabetes mellitus, multiple sclerosis, encephalomyelitis, inflammatory bowel disease, psoriasis, and pemphigus vulgaris.

20. The method of claim 18, wherein the immune system disorder, condition, or disease is an anti-inflammatory disorder selected from bacterial and parasitic infections, allergies and other atopic disorders, chronic graft versus host disease, scleroderma, and systemic lupus erythematosus.
